(19) United States
(12) Patent Application Publication
(10) Pub. No.: US 2005/0197544 A1
(43) Pub. Date: Sep. 8, 2005

(54) System and method for indexing emergency department crowding

(76) Inventor: Steven L. Bernstein, Closter, NJ (US)

Correspondence Address: OSTROLENK FABER GERB & SOFFEN, 1180 AVENUE OF THE AMERICAS, NEW YORK, NY, 100368403, US

(21) Appl. No.: 11/066,630

(22) Filed: Feb. 24, 2005

Related U.S. Application Data
(60) Provisional application No. 60/547,699, filed on Feb. 24, 2004.

(51) Int. Cl. G06F 17/60; A61B 5/00

(52) U.S. Cl. 600/300; 705/3

(57) ABSTRACT

A quantitative measure of emergency department (“ED”) crowding and busyness is provided to ED staff and physicians substantially in real time. Further, the present invention enhances patient safety and improves quality of care by alerting ED personnel to of crowding, who are able to prevent adverse outcomes typically caused by ED crowding.

### CROSS-REFERENCE TO RELATED APPLICATIONS

This patent application is based on and claims priority to U.S. Provisional Patent Application Ser. No. 60/547,699, filed on Feb. 24, 2004 and entitled SYSTEM AND METHOD FOR DEVELOPING AND VALIDATING AN INDEX TO MEASURE EMERGENCY DEPARTMENT CROWDING, the entire contents of which are incorporated herein by reference.

### BACKGROUND OF THE INVENTION

Hospital emergency department (“ED”) crowding is thought to be associated with adverse outcomes, such as errors in patient treatment and decreased patient satisfaction. While ED crowding is clearly related to ED volume, an ED can be busy without being “crowded.” As used herein, the term, “crowding” refers, generally, to a condition wherein the needs of the patients in the ED (and waiting room) exceed the capacity of the department. Also as used herein, an ED refers, generally, to the industry recognized definition as provided by the American College of Emergency Physicians (www.acep.org).

Although emergency medical physicians have an intuitive sense of when an ED is becoming crowded, there is no universally accepted quantitative measure or index of ED crowding, and ED crowding remains difficult to define. The existing literature on ED crowding is characterized by confusion between causes of crowding, outcomes of crowding, and actual measures of the phenomenon itself. Accordingly, ED crowding continues to result in errors in patient treatment in emergency departments in hospitals, acute care facilities, or specialty hospitals.

In the prior art, proposed identifiers of ED crowding have included the following: a transfer time of admitted patients from the ED to an inpatient bed of greater than 4 hours; all ED beds filled more than 6 hours per day; patients placed in hallway beds more than 6 hours per day; physicians feeling so rushed that they may make errors; ten or more patients per day who have waited more than 10 hours to be seen by the emergency physician; and more than 30% of ED beds filled with admitted patients who are awaiting an inpatient bed. Unfortunately, none of these prior art markers are exclusively accurate, given wide variations in ED size, patient visit volume, acuity of patient conditions, and staffing capacity found in U.S. EDs.

In the 1990s, U.S. hospital emergency departments experienced explosive growth in patient visits, concurrent with a sharp decline in the number of hospital EDs. According to data from the National Hospital Ambulatory Medical Care Survey, between 1992 and 2001, annual ED visit volume rose 23% from 89.8 million to 110.2 million. At the same time, the number of EDs fell from 5169 in 1988 to 4037 in 2002. FIG. 1 is a line graph that illustrates these trends. Crowding is, therefore, considered a function of patient volume, patient acuity, physical space, and on-duty staff. As increasing attention is being paid to EDs as a vital component of the nation's health care safety net, ED crowding has moved to the top of the policy agenda in emergency medicine.

Prior art descriptions of ED crowding focused on factors such as the growth in the number of substance users, homeless, AIDS patients and mentally ill in urban areas. A 1993 report by the U.S. General Accounting Office (“GAO”) attributed the growing volume of ED visits to these factors, noting that many of these patients were uninsured or had Medicaid, and were using the ED for nonurgent conditions. More visits by the elderly were noted as well. This report and others focused attention on nonurgent, or “unnecessary,” visits by uninsured and underinsured patients as the root cause of ED crowding. Unfortunately, this is misleading as the “root” cause of ED crowding.

In 2003, the GAO revisited the issue of ED crowding. In contrast to its 1993 study, this time the GAO found that the single greatest contributor to ED crowding was the prolonged presence in the ED of patients already admitted to hospital, for whom no inpatient bed is available. These patients, referred to herein, generally, as “boarders” and/or “holds,” require considerable amounts of nursing time, physician time, and medical resources. Moreover, by occupying ED beds or treatment bays, they also prevent new patients from being seen and treated. It has become apparent that reduction of ED crowding would necessarily involve more efficient management of “boarders,” including faster transfer to an inpatient unit. As used herein, an ED treatment bay refers, generally, to a care space or bed designed to hold one patient.

More recently, the first sustained policy, administrative, and clinical initiatives to cope with ED crowding have been proposed. This flurry of activity was spurred in part by the 2001 “Expert Meeting on ED Crowding and Ambulance Diversion,” convened by the US Department of Health and Human Services. These measures have met with some success, as episodes of ambulance diversion (closing an ED to arriving ambulances) became less frequent in 2002-2003 than in previous years.

In 2000, New York State Commissioner of Health and former U.S. Surgeon General Antonia Novello issued a directive to all acute care hospitals in the state, permitting the placement of admitted ED patients in “hallway” spaces of inpatient wards, if an inpatient room was unavailable. Although not yet universally implemented, anecdotal evidence suggests this directive has allowed the two hospitals in this study to relieve some ED congestion, with no apparent adverse clinical outcomes.

In 2002, the Robert Wood Johnson Foundation began a national program designed to identify practical solutions to ED crowding, and to assess the impact crowding has on health care safety. The program, known as Urgent Matters, awarded 10 grants to health care systems located in the United States. Early results from Urgent Matters suggest that a combination of hospital-wide policies reduces diversion and improves ED throughput. These benefits result from greater attention to inpatient discharge planning, faster turnaround from radiology and laboratory services, and greater coordination of care among EDs in a geographic region.

In 2003, the Joint Commission (“JCAHO”), a voluntary body that accredits and evaluates quality for hospitals and health care systems, issued landmark guidelines on crowding (available at wwwjcaho.org). For the first time, the JCAHO recognized the link between crowding and quality. Without mandating specific policies, these guidelines call for hospitals to have plans in place to handle a crowded ED, and to provide a level of service to admitted patients boarding in the ED comparable to that which they would receive on an inpatient unit.

Although measures are improving, analysis of ED crowding such as described above has been hampered by lack of a uniform definition. One of the prior art definitions of crowding includes periods when the ED is on diversion. Diversion occurs when patients who require emergency care are diverted from one ED facility to another because the first ED does not have the resources, for example, empty beds, to treat the patients. Other prior art definitions include times when daily visit totals exceed a predetermined threshold, and when all ED beds are filled for more than eight hours in a given day. Unfortunately, no prior art definitions of ED crowding have been validated, none are scaled for use by EDs of different sizes, and none have been used as a real-time measure to improve patient care.

### SUMMARY OF THE INVENTION

There is a need in the industry to develop a quantitative measure of ED crowding and busyness that can be provided to ED staff and physicians substantially in real time. There is further a need to provide a comparative measure to represent crowding among the different emergency departments.

The present invention comprises a measure of emergency department (ED) crowding, referred to herein, generally, as the Emergency Department Work Index (“EDWIN”). Using EDWIN, patient safety and quality of care in EDs is improved by providing a real-time measure of ED crowding that can predict when adverse events are likely to become more prevalent. More particularly, EDWIN is easily calculated using regularly collected and standard administrative data.

The present invention employs a conceptual model of ED crowding known in the art as the Input/Throughput/Output model, described by Asplin et al. The model explains ED crowding as resulting from increased numbers and/or acuity of arriving patients, inefficient ED operational processes, “downstream” obstacles to moving patients out of the ED or some combination of these factors.

The Input/Throughput/Output model is applied with reference to EDWIN. The present invention enhances patient safety and improves quality of care by providing a real-time measure of ED crowding that can predict when adverse events are likely to become more prevalent.

### BRIEF DESCRIPTION OF THE DRAWINGS

For the purpose of illustrating the invention, there is shown in the drawings a form which is presently preferred, it being understood, however, that the invention is not limited to the precise arrangements and instrumentalities shown. The features and advantages of the present invention will become apparent from the following description of the invention that refers to the accompanying drawings, in which:

FIG. 1 is a line graph that illustrates emergency department crowding trends;

FIG. 2 graphically illustrates a series of factors that contribute to ED crowding at varying points in the patient flow process;

FIG. 3 represents activity occurring in a hospital or other emergency department;

FIG. 4 illustrates an example patient medical record display screen in accordance with an example embodiment of the present invention;

FIGS. 5A and 5B illustrate display screens of an example ED clinical information system and that incorporate an EDWIN value in accordance with an example embodiment of the present invention; and

FIG. 6 illustrates an example columnar data report that represents ED clinical information data and EDWIN measurements in accordance with an example embodiment of the present invention.

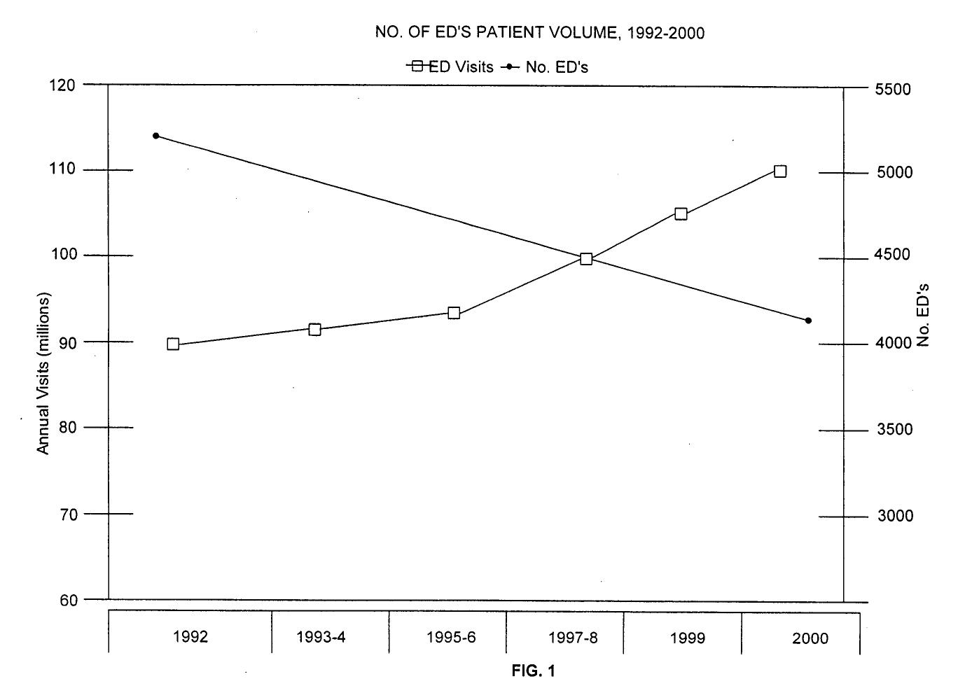

### DESCRIPTION OF THE EMBODIMENTS

The present invention provides a software mechanism comprising one or more modules that operate to alert hospital personnel when an ED is approaching critical crowding levels. As used herein, the term, “module,” refers, generally, to one or more discrete components that contribute to the effectiveness of the present invention. Modules can include software elements, including but not limited to functions, algorithms, classes and the like. Modules also include hardware elements, substantially as described below. Modules can operate independently or, alternatively, depend upon one or more other modules in order to function.

In a preferred embodiment of the present invention, a software application operates with an emergency department's existing software application(s), for example, a patient tracking system that enables physicians and ED staff to assess ED activity substantially in real time. Preferably, the software application provided by the present invention includes one or more graphic representations of ED crowding.

The present invention measures ED crowding in a “universal,” reproducible and consistently accurate way for EDs of different sizes. The present invention preferably provides a measurement or index that includes immediately available data elements from existing sources or that are continuously monitored by existing information systems. An additional feature of the present invention is the programmability of the measure into an ED's electronic patient tracking system, so that the measure is used as a real-time measure of crowding.

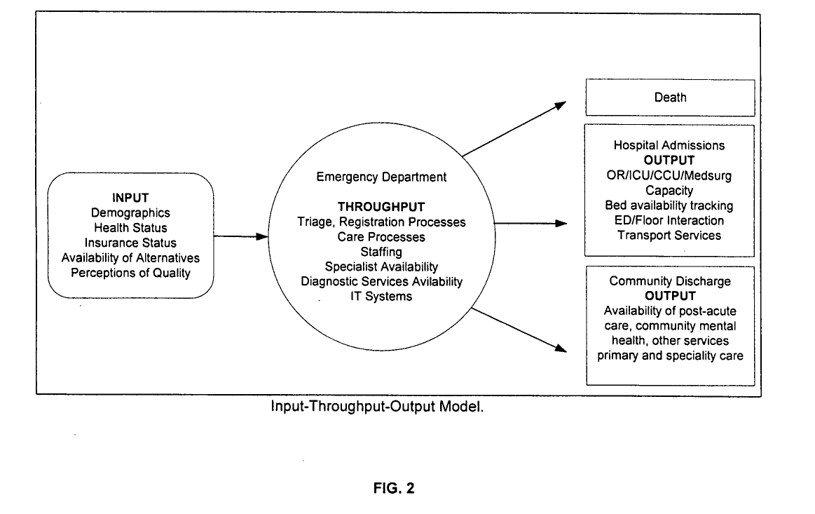

The index provided by the present invention, (i.e., the Emergency Department Work Index (“EDWIN”)), incorporates pertinent components of the Input/Throughput/Output model, including the number and acuity of patients, ED staffing, and bed availability, and provides the components in a single composite index. FIG. 2 graphically illustrates a series of factors that contribute to ED crowding at varying points in the patient flow process. The factors are divided into three domains, Input, Throughput, and Output, and correspond to their “sites of action.” In accordance with a preferred embodiment of the present invention, EDWIN is included (e.g., programmed) into a emergency department's patient tracking software as a real-time measure of crowding and provides an opportunity to use the relationships between ED crowding and adverse events, including medical error and deviations from evidence-based standards of care, in order to improve patient care.

The Emergency Department Work Index (EDWIN) is defined as follows: EDWIN=Σnᵢₜtⱼ/Nₜ(B−Bₜ). The variables in the EDWIN equation are as follows: ni=the number of patients present in the ED in triage category i at time t; tj=the triage score (ordinal scale 1-5, 5 being most acute) for the jth patient; Nt=the number of attending physicians on duty at time t; B=the total number of beds, or treatment bays, available in the ED (and is, typically, a constant); Bt=the number of admitted patients (holds) in the ED at time t.

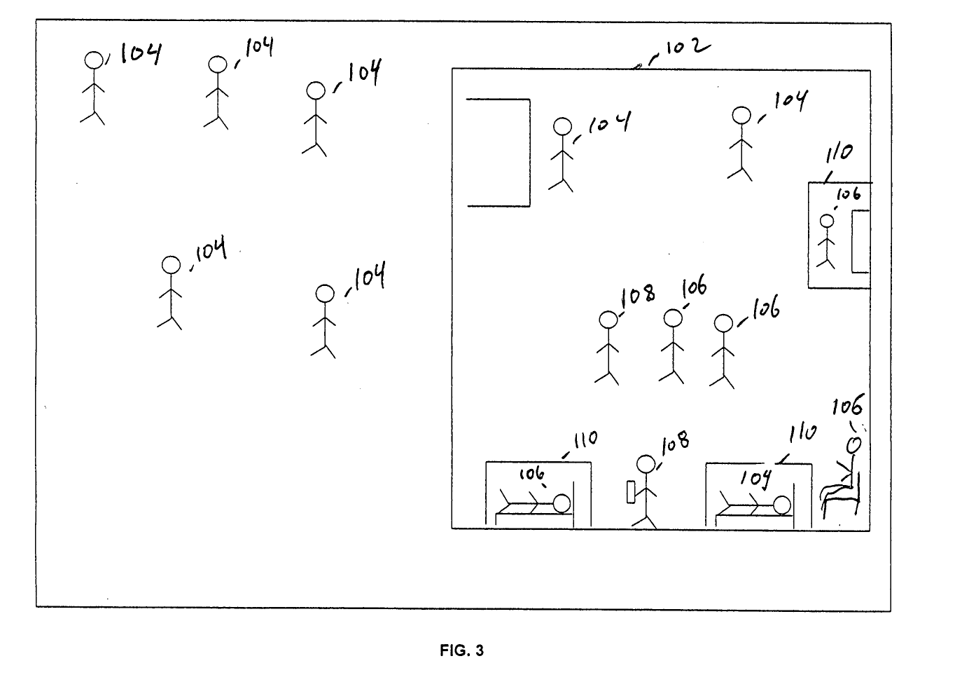

FIG. 3 represents activity occurring in a hospital or other ED 102. As shown in FIG. 3, patients 104 enter the ED 102 to be treated or seen by an ED caregiver. Patients 106 are patients who have been admitted and are awaiting transport to a bed (“holds”). Physicians 108 in the ED 102 provide medical care for patients 106, 108 who are placed in treatment bays 110.

As noted above, once a patient 106 is admitted, he/she is counted as a “hold,” and is removed from the numerator, which is calculated as a sum of the triage categories of all active patients 104 in the ED 102. The number of treatment bays (B) 110 is preferably derived from the ED's 102 original blueprint, or certificate of occupancy. Preferably, treatment bays 110 do not include the various beds creatively placed in hallways and corners by ED personnel. Treatment bays 110 also do not include the second bed in a room designed for one patient that has been “doubled up.”

The quantitative measure of ED 102 activity is correlated with adverse outcomes in order to achieve various goals and solve various problems. For example, a link correlating ED 102 crowding to patient quality of care and safety is provided by the present invention. Further, a plurality of emergency departments of varying sizes and capacities can be compared using uniform standards. Additionally, ED 102 crowding as generally conceived of, is broadened to include the wider context of overall levels of activity in the ED 102 that may be more accurate in order to analyze relationships between crowding, activity levels and adverse outcomes.

In a preferred embodiment of the present invention, a triage index system is applied that is the known Emergency Severity Index (ESI). The ESI is a five-level instrument that has high interobserver agreement, and is associated with resource use, hospitalization rates, and six-month survival. This particular instrument is used due to its reproducibility and simple, algorithmic quality. However, in accordance with a preferred embodiment of the present invention, the ESI is modified slightly by reversing the ordinal ranking of triage categories: that is, in the EDWIN use of ESI, 1 is the least acute patient (unlike prior art use of ESI wherein 1 is the most acute), and 5 is the sickest (unlike prior art ESI where 5 is the least acute). This was done to maintain the arithmetic sensibility of the index: as the numerator increases in value, the “busyness” of the ED 102 increases. Although in a preferred embodiment EDWIN uses ESI for the triage category, one skilled in the art will recognize that EDWIN can be adapted for other systems.

Hence, the units of EDWIN can be represented as “patient triage units per attending physician 108 per available bed 110.” Its numerator and denominator represent the ratio of ED 102 workload (e.g., patient triage scores) to ED 102 resources (staff and available beds 110). EDWIN is a dynamic index; its value may change from moment to moment.

The three components of the index reflect the three domains of the Input/Throughput/Output model (described above). The numerator, the sum of all patient triage scores, reflects Input. The number of physicians 108 on duty reflects throughput, as does the total number of ED beds 110. The number of admitted patients 106, or boarders, reflects Output; were beds available in inpatient units, the number of boarders 106 would be low, and the index smaller.

As known to those skilled in the art, attending physicians are not the only providers of care within EDs 102. For example, residents and so-called physician extenders (e.g., nurse practitioners and physician assistants) may examine and treat patients as well. In a preferred embodiment of the present invention, these groups are not represented in the index for different reasons. First, residents are typically not the sole physician to see any patient; because they are trainees, residents' patients are interviewed and examined by the attending physician, after the resident discusses the case with the attending. The contribution of residents to patient flow is unclear. Data are sparse, but a standard textbook of ED management states data suggests that residents generally prolong ED 102 patient length of stay. Furthermore, most EDs 102 do not employ residents. The present invention trying creates a measure of crowding applicable to all EDs 102, thus “house officers” are not included in a preferred embodiment. Of course, one skilled in the art will recognize that EDWIN may be modified to include residents and physician extenders to provide an index representing ED 102 crowding.

Unlike residents, physician extenders may operate independently in the ED 102. A variety of staffing models are used. In some institutions, the physician extender operates independently, perhaps with the attending co-signing the patient chart; in others, the physician extender functions much like a resident, presenting each patient to the attending physician for review and creation of a care plan. In EDs 102 where the physician extender works as an independent provider, the present invention preferably counts the physician extender as an attending physician 108, and contributes to the denominator of EDWIN. Alternatively, if the physician extender is working under the direct supervision of an attending physician 108, the physician extender is preferably not included in the index.

To examine whether EDWIN scores are higher for patients who suffer an adverse event, scores are preferably calculated for individual patients. A description of this process is provided below.

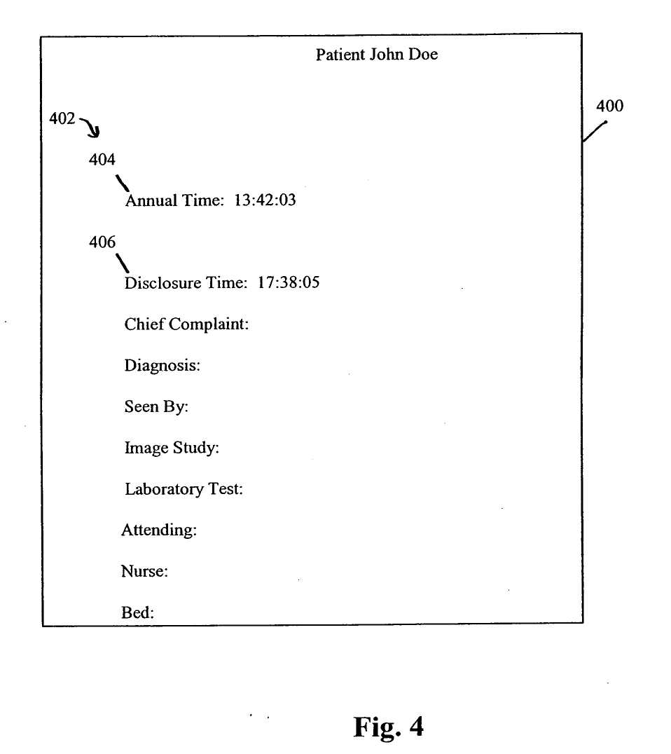

FIG. 4 illustrates an example patient medical record display screen. 400 comprising medical record 402. From the electronic medical record 402, patient arrival time 404 and patient discharge time 406 from the ED 102 are preferably extracted. Preferably, EDWIN scores measured during that patient's ED 102 stay is preferably averaged, and the mean EDWIN score obtained is assigned to that patient visit. In case a patient is treated and released during a period when EDWIN is not measured, a score is preferably obtained for that patient by averaging the scores immediately preceding and following that visit. (For example, if scores are measured at noon and 2 p.m., and a patient's ED 102 visit runs from 12:30-1:30 p.m., the noon and 2 p.m. scores are averaged. This reasonably approximates the level of crowding during the patient's ED 102 stay.)

In a preferred embodiment of the present invention, score averaging and assignment is performed using SAS (version 9.0 or other current version), with data extracted from the electronic patient records of the hospital (or other) ED 102.

It is envisioned herein that a plurality of uses of EDWIN are available. For example, a software application operates with an emergency department's computer hardware and software for, for example, patient tracking, and enables physicians and ED 102 staff to assess ED 102 activity substantially in real time. Preferably, the software application provided by the present invention includes one or more graphic representations for representing ED 102 crowding. In a preferred embodiment, the EDWIN Since increasing EDWIN scores are associated with adverse outcomes and medical error, then a real-time “dashboard” function of the score may be used to warn ED 102 staff and hospital administrators when adverse outcomes are about to become more common.

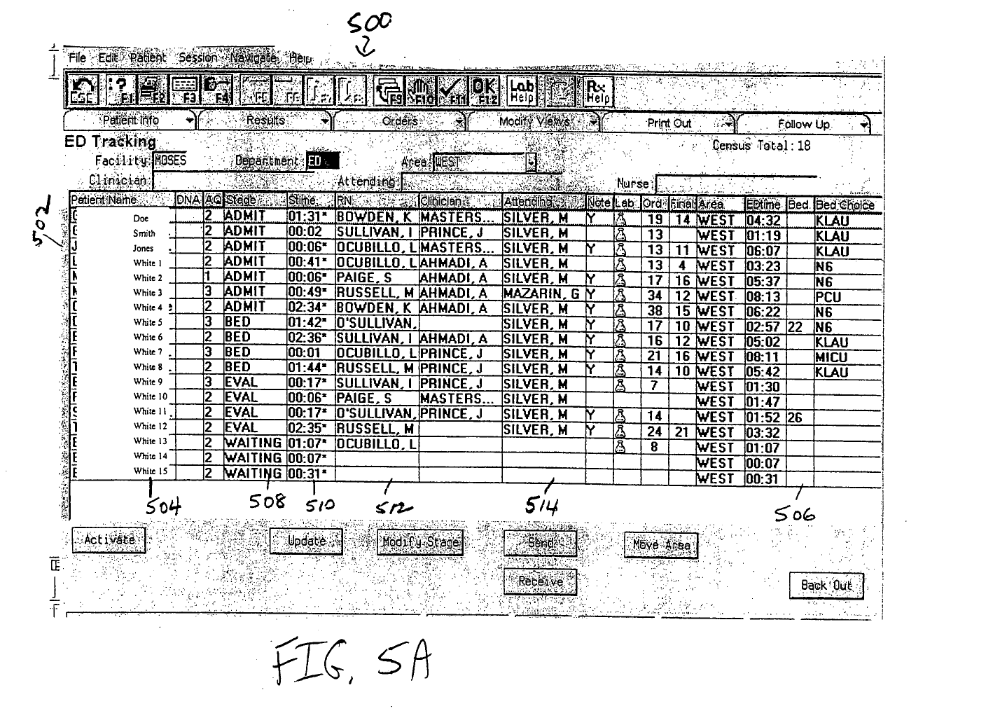

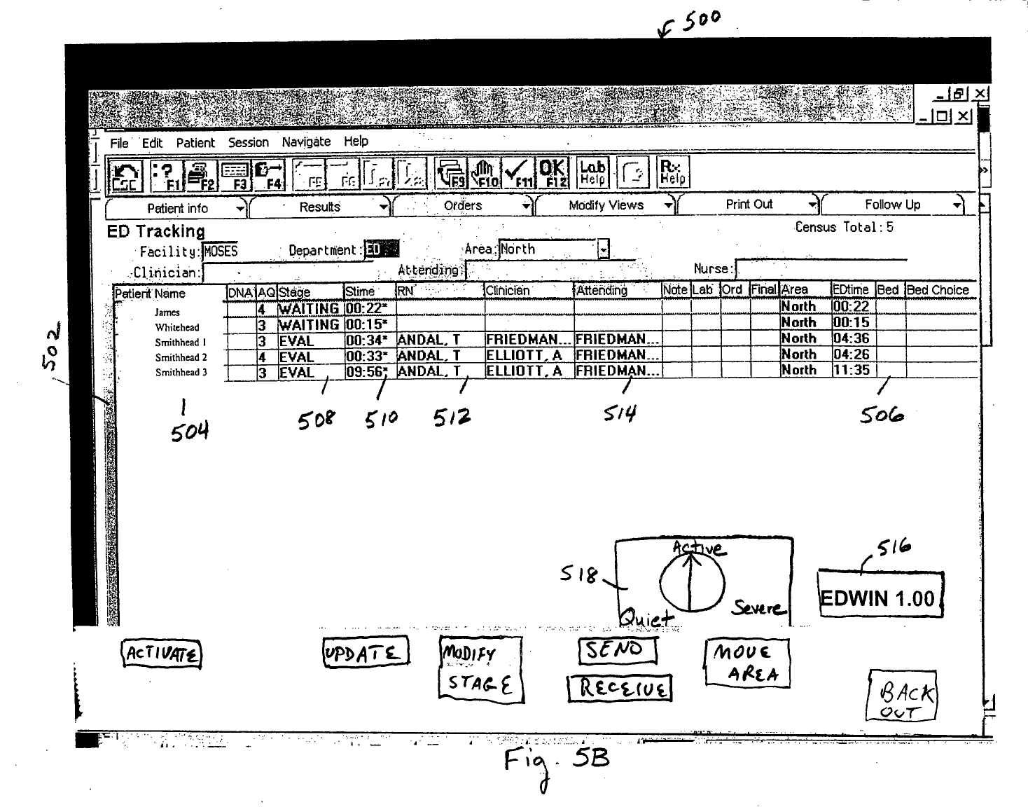

FIGS. 5A and 5B illustrate display screens 500 of an example ED 102 clinical information system 502. These systems may vary slightly in terms of capability and functionality, but often have commonality. Common elements that are included in many clinical information systems include patient tracking and computerized order entry for imaging studies and laboratory tests. Typically, patient tracking system 502 provides a real-time screen listing 504 the names of all patients in the ED 102, their bed location 506, whether they are admitted 508, their length of stay 510, the nurse name 512 and the physician name 514 and caring for each patient. The tracking system 502 is, typically, used routinely by all ED 102 staff.

FIG. 5B illustrates clinical information system 502 with an EDWIN score programmed therewith and illustrated in EDWIN section 516. As shown in FIG. 5B, the EDWIN score is 1.0, which is considered active but manageable, and alerts ED personnel, including staff and physicians, thereof. Also as shown in FIG. 5B, gauge 518 indicates the ED 102 at an active state.

For policymakers, researchers, and administrators, EDWIN scores provide an important and independent covariate in understanding the causes of medical error and adverse quality of care caused by crowding. If EDs 102 have multiple components (for example, multiple treatment areas or zones), then EDWIN preferably provides scores of those individual components to determine where a new patient should be assigned. Furthermore, for EDs 102 in a single jurisdiction, ambulance diversion decisions could be made by offering diversion to the ED 102 with the highest EDWIN score.

In one embodiment of the present invention, resources including, for example, capacity of an emergency department to treat ED patients 104, can be increased or decreased in accordance with conclusions in part drawn by the index of the present invention. Further, information relating to overcrowding in the emergency departments can be transmitted to patient transport systems in order to provide a more equitable distribution of patients among a plurality of emergency departments. For example, ambulances and other patient transport vehicles preferably use wireless devices to receive information from the present invention to provide accurate and uniform measurements of patient crowding in a plurality of emergency departments substantially in real time. By utilizing the software provided by the present invention in a plurality of contexts, patient clinical outcomes improve. For example, the software application provided by the present invention includes a representation of the Emergency Department Work Index to provide a convenient measure of ED 102 busyness and crowding. Further, the present invention provides a useful comparative tool for a plurality of emergency departments of varying sizes and capacities.

As shown in FIG. 5B, a software application is preferably provided that allows parties operating in emergency departments to monitor the degree of patient crowding at any given time. In an embodiment of the present invention, a series of display screens are preferably provided automatically periodically to gather information from emergency department staff and physicians in order to maintain updated information and provide accurate representations of emergency department crowding. Preferably, the software application of the present invention is integrated in an emergency department's computer hardware and software environment such that emergency department staff and physicians are alleviated from having to remember to start the application to enjoy the benefits the present invention provides. By seamlessly integrating the software application of the present invention and existing software applications in an emergency department computing environment, data is entered more easily and accurate and updated representations of emergency department crowding are regularly provided for the user.

It is envisioned herein that the software application provided by the present invention is installed on a plurality of workstations and in a plurality of emergency departments. The responses and additional data collected by the software application are preferably compiled to a central information processor, such as a computer server, such that responses from a plurality of users can be analyzed and manipulated to provide an accurate assessment of the degree of crowding in several emergency departments at a given time.

In addition to one or more display screens provided for receiving information from emergency department staff and physicians, a graphical interface is preferably provided that informs the user of the degree of crowding of the emergency department at any time. For example, a gauge, such as commonly found in a motor vehicle, is displayed on a computer display to indicate the status of the emergency department. One skilled in the art will recognize that such a representation may take many different forms. In one example embodiment, a round gauge is provided with a needle that points to position in the circumference of the gauge that represents the degree of crowding. Preferably, a portion of the gauge is shown in a different color from the rest of the gauge, for example, red, to indicate crowding has reached critical levels. In another alternative embodiment, a gauge is provided that is a vertically oriented rectangle. A line or other indicator is included and positioned within the rectangle to indicate the degree of crowding in the emergency department. In yet another alternative embodiment, only a number is provided that represents the degree of overcrowding. The number can, preferably, change colors depending upon a predetermined range. For example, a number between 0 and 1.5 is presented in green, a number between 1.5 and 2 is presented in yellow, and any number over 2 is presented in bright red.

Of course, many other types of interfaces and representations can be provided, as one skilled in the art will recognize, in order to identify for an emergency department staff or physician the degree of crowding and emergency of the emergency department at that time.

As noted above, information regarding the degree of crowding in the emergency department at any given time is shared among a plurality of parties, such as patient transport units, on-site and off-site physicians, emergency department administrators, and other healthcare facilities. In an example embodiment of the present invention, an information processor, such as a computer system operating as a server, receives information collected and compiled by the software application of the present invention from a plurality of emergency departments in a geographic region.

For example, an emergency department at one hospital may be experiencing severe crowding due to a crisis occurring in close proximity to that emergency department. Another emergency department, located farther from the emergency scene, is not experiencing the same degree of crowding. In accordance with the present invention, a comparison can be made automatically made by applications operating on the server computer system, and patients on route to the overcrowded emergency department can be diverted to the emergency department not experiencing the heavy degree of crowding. In this way, resources among a plurality of emergency departments can be more equally distributed among a geographic region, and patient care can improve as a result.

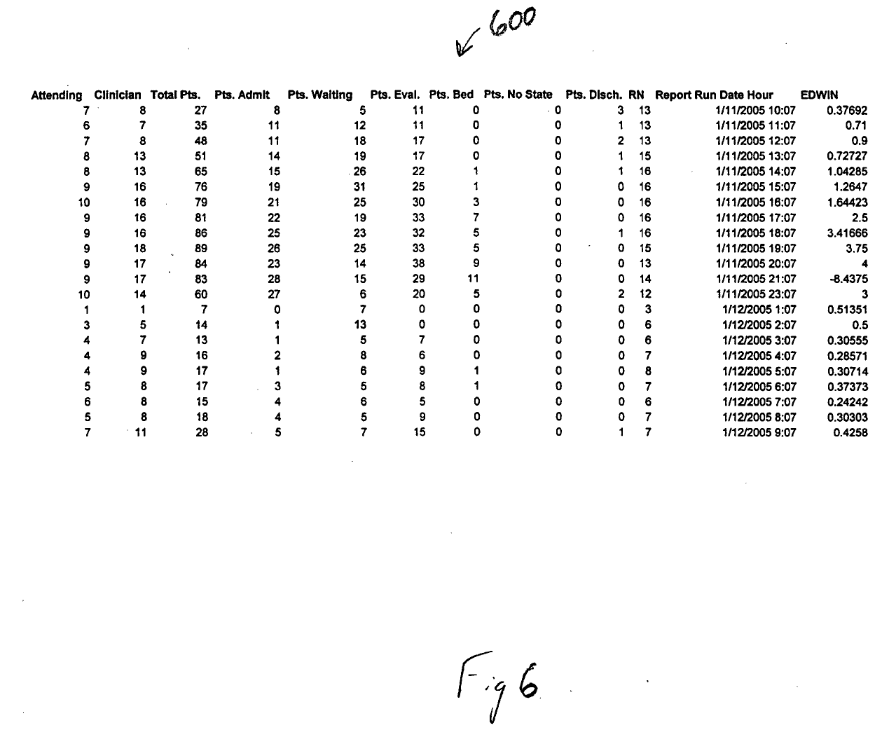

FIG. 6 illustrates an example columnar data report 600 that represents ED 102 clinical information data and EDWIN measurements provided by an example embodiment of the present invention. As shown in FIG. 6, each row represents information collected in an ED 102 at a given time. For example, the first row of data represents the number of attending physicians 108, clinicians, patients 104, patients who are admitted, patients waiting 106, patients evaluated, patient beds 110, patients no state, patients discharged, registered nurses and a corresponding EDWIN value taken at 10:07 in the morning. The second row of data comprises the same categories of information taken at 11:07. Thus, the report provides information representing the above-listed categories every hour during a day. In this way, ED 102 crowding can be monitored over time in a concise format.

A discussion regarding evidence directed to the present invention is provided below.

The Emergency Department Work Index is highly correlated with ED 102 crowding as measured by physician and nurse assessments. To assess the strength of association between EDWIN and evidence-based processes of care, the strength of association between EDWIN and a series of quality endpoints is examined.

EDWIN has been tested in several EDs 102. EDWIN has shown good correlation with nurse/physician assessment of crowding at eight EDs 102 in the Emergency Department Crowding Score study. Preliminary data has suggested that EDWIN is a useful measure of crowding, with both construct and content validity.

Further, a strong association has been found between EDWIN scores and ambulance diversion, one of the known consequences of ED 102 crowding.

EDWIN testing has suggested that ED 102 activity may be demarcated into three ordinal zones: an active but manageable ED 102 has an EDWIN score<1.5; a busy ED 102 as an EDWIN between 1.5 and 2, and a crowded ED has a score>2. These three zones, active, busy and crowded, suggest an “ABC” paradigm to characterize ED activity.

An alternative embodiment of the present invention is now provided with respect to incorporating nursing staff as a variable in the present invention.

When an ED 102 is busy, nurse and physician staffing are maximal; the converse is also true when the ED 102 is “quiet.” Thus, nurse staffing fluctuates frequently during the course of a shift. At times, nurses assigned to direct patient care may be reassigned to the triage area to assist with increased patient arrivals; nurse breaks are lengthy enough to have a measurable impact on staffing (in New York state, 1.5 hours of break during a 12-hour shift).

In an alternative embodiment of the present invention, EDWIN's predictive ability is provided when nurse staffing is incorporated. Due to a current and worsening condition related to a shortage of nurses, many EDs 102 are often understaffed either chronically (unfilled staff positions) or intermittently (shifts without adequate nurses present). Given these limitations, nurse staffing may not always mirror physician staffing. Nurses make important contributions to ED patient 104 throughput, independent of the work of the physician. Nurses perform triage of newly arrived patients (in most EDs 102), some elements of patient registration, and numerous processes of care that are labor-intensive. In many EDs 102, nurses handle the patient discharge process, reviewing follow-up and treatment instructions with patients at the close of their treatment. In addition, nurses continue to provide routine care for boarding patients, who are admitted but waiting for an inpatient bed. This is in contrast to the attending physician, who typically transfers care of the admitted patient to the inpatient physician, even if the patient is still in the ED 102.

A small but growing body of evidence suggests that higher nurse/patient ratios result in higher mortality rates and adverse performance on quality measures, for inpatients, critical care unit patients, and residents of nursing homes. Accordingly, an alternative embodiment of EDWIN is provided as follows:
EDWIN_{modified}=Σnᵢₜtⱼ/NₜNₙ(B−Bₜ)

The variables in the alternative (or modified) EDWIN equation are as follows: Nn=the number of nurses on duty at a given time (this number comprises nurses who are assigned to direct patient care, because their names are captured in patients' electronic medical records. Supervisory nurses and triage nurses, who do not typically have direct responsibility for individual patients, are preferably not represented in the modified index.) By including nurses, this version of the index more fully represents the important domains of the Input/Throughput/Output model. The modified EDWIN does, of course, have different units and values than that described previously.

Other features and benefits of the present invention are further envisioned. For example, in addition to routing patients from facilities experiencing overcrowding to those that are not, a present invention provides for accurate comparative analysis of a plurality of emergency departments at given time periods and over time. Using the methods described herein, emergency departments of varying sizes and resources can be compared in order to draw conclusions based upon crowding and patient care. Other benefits will be apparent to one skilled in the art.

Although the present invention has been described in relation to particular embodiments thereof, many other variations and modifications and other uses will become apparent to those skilled in the art. Therefore, the present invention is not limited by the specific disclosure.

1. A method for providing a measurement representing an assessment of emergency department (“ED”) crowding in a medical facility, the method comprising:
receiving electronic patient information, said electronic patient information is representative of a plurality of patients who are in said ED;
storing said electronic patient information in a database;
receiving electronic patient caregiver information, said electronic patient caregiver information is representative of at least one person providing treatment to at least one of said plurality of patients;
storing said electronic patient caregiver information in said database;
receiving electronic treatment bay information, said electronic treatment bay information is representative of treatment bays in said ED;
storing said electronic treatment bay information in said database;
receiving electronic patient treatment information, said electronic patient treatment information comprising, for each of said plurality of patients, at least an arrival time at said ED, a triage category value, and a value indicating whether each of said plurality of patients has been admitted to said medical facility;
storing said electronic patient treatment information in said database;
using said electronic patient information, said electronic patient caregiver information, said electronic treatment bay information and said electronic patient treatment information to calculate said measurement that represents crowding in said ED; and
providing said measurement.

2. The method of claim 1, wherein the step of calculating comprises Σnᵢₜtⱼ/Nₜ(B−Bₜ), wherein:
nᵢ represents a number of said patients present in said ED in triage category i at time t;
tⱼ is representative of a triage score for a jth patient;
Nₜ is representative of a number of attending physicians on duty at time t;
B is representative of the available treatment bays; and
Bₜ is representative of the number of holds in said ED at time t.

3. The method of claim 2, wherein said triage score is an Emergency Severity Index ordinal score ranging from 1 to 5, wherein 5 is representative of a most acute patient condition.

4. The method of claim 1, wherein the step of providing said measurement comprises integrating said measurement with a software application utilized by said ED for patient tracking.

5. The method of claim 1, wherein said providing said measurement comprises displaying a graphical representation of ED crowding.

6. The method of claim 5, wherein said graphical representation is representative of a degree of ED crowding and is displayed as at least one selected from the group consisting of a gauge, a chart, a graph, and the measurement displayed in a color, wherein the color is representative of a degree of ED crowding.

7. The method of claim 1, wherein the steps of receiving and storing electronic patient information, electronic patient caregiver information, electronic treatment bay information, and electronic patient treatment information, and calculating said measurement are repeated during the course of a day.

8. The method of claim 1, wherein the step of providing said measurement further comprises alerting ED personnel when said ED crowding is approaching critical levels.

9. The method of claim 1, further comprising modifying treatment of patients in said ED based on said measurement, wherein said step of modifying treatment comprises at least one of diverting patients to a different ED that is less crowded and adjusting the number of medical caregivers in said ED.

10. The method of claim 1, further comprising providing at least report that summarizes crowding of said ED over the course of time.

11. The method of claim 1, wherein the steps of receiving said electronic patient information, said electronic patient caregiver information, said electronic treatment bay information and said electronic patient treatment information occurs substantially automatically from a patient tracking software application.

12. The method of claim 1, wherein the step of calculating comprises Σnᵢₜtⱼ/NₜNₙ(B−Bₜ), wherein:
nᵢₜ is representative of a number of said patients present in said ED in triage category i at time t;
Nₙ is representative of a number of nurses on duty at a given time;
tⱼ is representative of a triage score for a jth patient;
Nₜ is representative of a number of attending physicians on duty at time t;
B is representative of the available treatment bays; and
Bₜ is representative of the number of holds in said ED at time t.

13. A system for providing a measurement that represents a relative assessment of emergency department (“ED”) crowding in a medical facility, the system comprising:
an electronic patient information module that electronically receives and stores in a database electronic patient information that is representative of a plurality of patients who are in said ED;
an electronic patient caregiver information module that electronically receives and stores in said database electronic patient caregiver information that is representative of at least one person providing treatment to at least one of said plurality of patients;
an electronic treatment bay information module that electronically receives and stores in said database electronic treatment bay information that is representative of treatment bays in said ED;
an electronic patient treatment information module that electronically receives and stores in said database electronic patient treatment information comprising, for each of said plurality of patients, at least an arrival time at said ED, a triage category value, and a value indicating whether each of said plurality of patients has been admitted to said medical facility;
a measurement calculation module that uses said electronic patient information, said electronic patient caregiver information, said electronic treatment bay information and said electronic patient treatment information to calculate said measurement that represents crowding in said ED; and
a display module that provides said measurement.

14. The system of claim 13, wherein said measurement is calculated using a formula: Σnᵢₜtⱼ/Nₜ(B−Bₜ), wherein:
nᵢ represents a number of said patients present in said ED in triage category i at time t;
tⱼ is representative of a triage score for a jth patient;
Nₜ is representative of a number of attending physicians on duty at time t;
B is representative of the available treatment bays; and
Bₜ is representative of the number of holds in said ED at time t.

15. The system of claim 14, wherein said triage score is an ordinal Emergency Severity Index score ranging from 1 to 5, wherein 5 is representative of a most acute patient condition.

16. The system of claim 15, wherein said display module integrates said measurement with a software application utilized by said ED for patient tracking.

17. The system of claim 13, wherein said display modules displays said measurement as a graphical representation of ED crowding.

18. The system of claim 17, wherein said graphical representation is representative of a degree of ED crowding and is displayed as at least one selected from the group consisting of a gauge, a chart, a graph, and the measurement displayed in a color, wherein the color is representative of a degree of ED crowding.

19. The system of claim 13, wherein the measurement calculation module repeats its operations during the course of a day.

20. The system of claim 13, wherein the display module alerts ED personnel when said ED crowding is approaching critical levels.

21. The system of claim 13, further comprising a treatment modification module that recommends modifying treatment of patients in said ED based on said measurement, wherein treatment modifying comprises at least one of diverting patients to a different ED that is less crowded and adjusting the number of medical caregivers in said ED.

22. The system of claim 13, further comprising a report module that provides at least hard copy report that summarizes crowding of said ED over the course of time.

23. The system of claim 13, wherein said measurement is calculated using a formula: Σnᵢₜtⱼ/NₜNₙ(B−Bₜ), wherein:
nᵢₜ is representative of a number of said patients present in said ED in triage category i at time t;
Nₙ is representative of a number of nurses on duty at a given time;
tⱼ is representative of a triage score for a jth patient;
Nₜ is representative of a number of attending physicians on duty at time t;
B is representative of the available treatment bays; and
Bₜ is representative of the number of holds in said ED at time t.
